SEQUENCE LISTING (17 sequences)

SEQ ID NO: 1 - protein, 230 aa, Homo sapiens
MEPLRLLILLFVTELSGAHNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLADPLDHRDAGDLWFPGESESFEDAHVEHSISRSLLEGEIPFPPTSILLLLACIFLIKILAASALWAAAWHGQKPGTHPPSELDCGHDPGYQLQTLPGLRDT

SEQ ID NO: 2 - protein, 11 aa, synthetic construct
RASQSVSSNLA

SEQ ID NO: 3 - protein, 7 aa, synthetic construct
GASTRAT

SEQ ID NO: 4 - protein, 9 aa, synthetic construct
LQDNNFPPT

SEQ ID NO: 5 - protein, 4 aa, synthetic construct
SWIG

SEQ ID NO: 6 - protein, 17 aa, synthetic construct
IIYPGDADARYSPSFQG

SEQ ID NO: 7 - protein, 12 aa, synthetic construct
RRQGIFGDALDF

SEQ ID NO: 8 - protein, 214 aa, synthetic construct
EIVMTQSPATLSVSPGERATLSCRASQSVSSNLAWFQQKPGQAPRLLIYGASTRATGIPARFSGSGSGTEFTLTISSLQPEDFAVYYCLQDNNFPPTFGQGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 451 aa, synthetic construct
EVQLVQSGAEVKKPGESLKISCKGSGYSFTSYWIGWVRQMPGKGLEWMGITYPGDADARYSPSFQGQVTISADKSISTAYLQWSSLKASDTAMYFCARRRQGIFGDALDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 17 aa, synthetic construct
KSSQSLLYSSNQKNYLA

SEQ ID NO: 11 - protein, 7 aa, synthetic construct
WASTRES

SEQ ID NO: 12 - protein, 9 aa, synthetic construct
QQYYNYPFT

SEQ ID NO: 13 - protein, 5 aa, synthetic construct
DYNIH

SEQ ID NO: 14 - protein, 16 aa, synthetic construct
YIYPKNGGTGYTQKFK

SEQ ID NO: 15 - protein, 10 aa, synthetic construct
RTARASWFAF

SEQ ID NO: 16 - protein, 119 aa, synthetic construct
EVQLVQSGAEVKKPGESLKISCKGSGYTFTDYNIHWVRQMPGKGLEWMGYIYPKNGGTGYTQKFKSQVTISVDNSISTAYLQWSSLKASDTAMYYCARRTARASWFAFWGQGTLVTVSS

SEQ ID NO: 17 - protein, 113 aa, synthetic construct
DIVMTQSPATLSVSPGERATLSCKSSQSLLYSSNQKNYLAWYQQKPGQAPRVLIYWASTRESGIPARFSGSGSGTEFTLTISSLQSEDFAVYYCQQYYNYPFTFGQGTKLEIK

(19) United States
(12) Patent Application Publication
(10) Pub. No.: US 2026/0078181 A1
(43) Pub. Date: Mar. 19, 2026

(54) TREM-2 AGONISTS FOR THE TREATMENT OF MARFAN SYNDROME

(71) Applicants: INSTITUT NATIONAL DE LA SANTÉ ET DE LA RECHERCHE MÉDICALE, Paris (FR); ASSISTANCE PUBLIQUE-HÔPITAUX DE PARIS (APHP), Paris (FR); SORBONNE UNIVERSITÉ, Paris (FR); UNIVERSITÉ PARIS CITÉ, Paris (FR)

(72) Inventors: Hafid AIT-OUFELLA, Paris (FR); Rida ALRIFAI, Paris Cedex 15 (FR)

(21) Appl. No.: 19/105,898

(22) PCT Filed: Sep. 5, 2023
(86) PCT No.: PCT/EP2023/074323

§ 371 Date: Feb. 24, 2025

(30) Foreign Application Priority Data
Sep. 6, 2022 (EP) 22306316.5

(51) Int. Cl. C07K 16/28

(52) U.S. Cl. CPC C07K 16/2803; C07K 2317/75

(57) ABSTRACT

Marfan syndrome is caused by mutations in the FBN1 gene (15q21) that codes for Fibrillin-1, an essential connective tissue protein and is a pathology responsible for a high morbidity and mortality. Apart from surgery, treatment options are limited. It is therefore essential to develop new pharmacological approaches to limit aortic dilatation and/or rupture. The inventors have demonstrated a critical role for TREM-2 in the pathophysiology of ascending aortopathy related to Marfan disease. Deletion of TREM-2 indeed aggravates ascending aorta dilation and rupture. Stimulating TREM-2 receptor with peptide or agonistic monoclonal antibody represent a new therapeutic approach for Marfan syndrome.

### FIELD OF THE INVENTION

The present invention is in the field of medicine, in particular vascular diseases.

### BACKGROUND OF THE INVENTION

Marfan syndrome is caused by mutations in the FBN1 gene (15q21) that codes for Fibrillin-1, an essential connective tissue protein. Border forms are secondary to mutations in the TGFBR2 gene located on chromosome 3, which codes for TGF-beta receptor. Its prevalence is estimated at 1/5000, i.e. 12,000 patients in France. Transmission is autosomal dominant. Thus, the disease affects both genders indiscriminately and an affected person has a 50% risk of transmission of the mutation. Symptoms can appear at any age and vary greatly from one person to another, even within the same family. Skeletal signs are often warning signs and may include dolichostenomelia (excessive length of the extremities), tall stature, arachnodactyly, joint hypermobility, etc. Ophthalmologic damage includes axial myopia which can lead to retinal detachment and displacement of the lens (ectopy or dislocation, a characteristic sign). There may also be skin signs (stretch marks), a risk of pneumothorax, and dural ectasia. More importantly, it is the cardiovascular disorders that conditions the prognosis of patients with Marfan syndrome with progressive dilatation of the ascending aorta accompanied by a high risk of a potentially fatal aortic dissection. Mitral valve (prolapse) or aortic valve abnormalities of the bicuspid type are also described. Pregnancy increases the risk of complications and should therefore be carefully monitored (Keane M G, Pyeritz, Circulation, 2008). Much progress has been made (Pyeritz et al, Heart 2009) in the management of Marfan patients but morbi-mortality remains too high.

The only treatment now recommended by experts is a beta-blocker, propranolol, which limits aortic dilatation and the risk of dissection (Shores et al, New Engl J Med 1994; Ladouceur et al, Am J Cardiol 2007). This treatment is recommended upon confirmation of the diagnosis of Marfan syndrome or related syndrome in case of aortic dilatation, or without aortic dilatation from the age of 4 years in the presence of a mutation. Medical treatment should be continued even after heart surgery and during pregnancy. It should not be stopped after childbirth (Omnes et al, Int J Gynaecol Obstet. 2013). In women with Marfan syndrome pregnancy is associated with an over-risk of aortic dissection. Above 45 mm aortic diameter, pregnancy is contraindicated. Type 1 angiotensin receptor blockers have been evaluated but their beneficial impact remains controversial. In an animal model of Marfan's disease, Losartan, an AT1R antagonist, improves arterial wall architecture and reduces aortic dilatation (Hibashi et al, Science, 2006). On the other hand, the interest of this molecule in humans is less clear. A randomized, double-blind, multicenter study reported no benefit (Milleron et al, Eur Heart J) while another study with a comparable methodology (N=192) reported a benefit of Losartan on the progression of aortic dilatation (Mullen et al. Lancet 2019). Surgical (+/− endovascular) treatment of the ascending aorta, alone or associated with a procedure on the aortic valve, is considered when the diameter of the ascending aorta is greater than 50 mm or when the increase in dilatation is rapid (more than 3 mm in one year, verified by 2 techniques), (adapted from the recommendations of the European Society of Cardiology 2014 (Erbel et al, Eur Heart J 2014). Surgical procedures are associated with significant perioperative complications such as leakage or dissection on the distal portion of the anastomosis.

In summary, Marfan Syndrome is a pathology responsible for a high morbidity and mortality. Apart from surgery, treatment options are limited. It is therefore essential to develop new pharmacological approaches to limit ascending aorta dilatation and/or rupture.

The pathophysiological mechanisms causing aortic dilatation and dissection in Marfan syndrome are not clearly understood. Marfan Syndrome is the consequence of a quantitative +/− qualitative genetic deficiency in Fibrillin-1. Fibrillin-1 is a glycoprotein present in large quantities in the extracellular matrix, it ensures tissue elasticity, an important mechanism to regulate the biomechanical stresses related to aortic ejection at the aortic root level (Dingemans et al Anat Rec. 2000). Fibrillin binds extracellular matrix proteins such as elastin and participates in the organization of microfibrils. In the aorta of patients with Marfan, the structure of the aortic wall is disorganized with breaks in the elastic blades and a scarcity of smooth muscle cells. The muscle cell phenotype is also modified, with overexpression of genes encoding contractile proteins (Crosas-Molist et al, ATVB 2015). The rate and activity of TGF-b is increased but several recent studies suggest that it is a compensatory and non-causal mechanism in aorta disease (Mallat et al, Circ Res).

Few pathophysiological work has evaluated the involvement of the immuno-inflammatory response in Marfan syndrome. However, several elements argue for a pathogenic role of immunity in the progression and complications of the disease. First of all, in the blood of Marfan patients, the level of M-CSF is higher in those with high aortic dilatation rates. In aortic media and aortic adventitia, there is a significant increase in T-cell and CD68+ macrophage infiltration in Marfan patients compared to aorta of control subjects. (Radonic et al PlosOne 2012) (D'amico, Int J Mol Sci. 2020; He, J Thorac Cardiovasc Surg 2006). In mouse models mimicking Marfan syndrome, there is also infiltration of inflammatory cells into the aortic wall with local overexpression of genes encoding chemokines (CCL-2, CCL-5), chemokine receptors (CX3CR1) and cytokines (IL-1b). However, the mechanisms that regulate the recruitment and activation of these macrophages are unknown, as is their involvement in vascular disease.

TREMs, which were identified as the new activating receptors of immunoglobulin superfamily expressed on human myeloid cells in 2000, include inhibitory and activating isoforms encoded by a gene cluster linked to the major histocompatibility complex (Bouchon, J Immunol 2000; Colonna, Nat Rev Immunol 2003). Currently, studies had explored several members of TREM family proteins including TREM1 (also known as CD354), TREM-2, TREM3, TREM4, plasmacytoid dendritic cell (pDC)-TREM, TREM-like transcript (TLT-1) and TLT-2. Among them, TREM-2 was an immunosuppressive receptor, and it has successfully attracted the attention of oncologists in recent years. Studies have shown that TREM-2 is expressed in some myeloid cells including DCs, monocytes, osteoclasts, Kuppfer cells, alveolar macrophages and microglia (Qi, Front Immunol 2021). To dates, studies have shown that TREM-2 has several biological functions, including but not limited to cell maturation, cell proliferation, cell survival, phagocytosis and the regulation of inflammation (Deczkowska Cell 2020). Once TREM-2 ligands bind to TREM-2, TREM-2 will interact with the adaptor proteins DAP12 and DAP10. The main kinase recruited by the ITAM region of DAP12 is spleen tyrosine kinase (SYK), which activates downstream signaling molecules such as PI3K, Akt, mTOR, MAPK, ultimately leading to cell activation, cell survival and the increase level of intracellular calcium (Mocsai, Nat Rev Immunol 2010). Beyond the above signalling pathway, TREM-2 also negatively regulates toll-like receptor (TLR) signalling pathways which play crucial roles in the innate immune system by recognizing pathogen-associated molecular patterns (Kawasaki, Front Immunol 2014). Long et al. found that TREM-2 could attenuate neuroinflammation through downregulating TLR signalling pathway (Long Neurochem Res 2019). Recently, Binder's group has reported that Trem2 deletion induced an increase of chemokines and pro-inflammatory cytokines production in a model of NASH and aggravates the liver disease (Hendrikx, J Hepatol 2022). Recently, agonistic activity of anti-TREM-2 antibodies have been described (Price, B. R., Sudduth, T. L., Weekman, E. M, Johnson, S., Hawthorne, D., Woolums, A., & Wilcock, D. M (2020). Therapeutic Trem2 activation ameliorates amyloid-beta deposition and improves cognition in the 5XFAD model of amyloid deposition. Journal of Neuroinflammation, 17(1), 238; Schlepckow, K., Monroe, K. M, Kleinberger, G., Cantuti-Castelvetri, L., Parhizkar, S., Xia, D., . . . Haass, C. (2020). Enhancing protective microglial activities with a dual function TREM-2 antibody to the stalk region. EMBO Molecular Medicine, 12(4), e11227; Wang, S., Mustafa, M., Yuede, C. M., Salazar, S. V., Kong, P., Long, H., . . . Colonna, M. (2020). Anti-human TREM-2 induces microglia proliferation and reduces pathology in an Alzheimer's disease model. Journal of Experimental Medicine, 217(9), e20200785.). The role of TREM-2 in Marfan syndrome and the interest of agonistic TREM-2 antibodies for the treatment of said disease have never been investigated.

### SUMMARY OF THE INVENTION

The present invention is defined by the claims. In particular, the present invention relates to the use of TREM-2 agonists for the treatment of Marfan Syndrome.

### DETAILED DESCRIPTION OF THE INVENTION

The first object of the present invention relates to a method of treating Marfan Syndrome in a patient in need thereof comprising administering a therapeutically effective amount of a TREM-2 agonist.

As used herein, the term “Marfan Syndrome” has its general meaning in the art and refers to a systemic disease of connective tissue characterized by a variable combination of cardiovascular, musculo-skeletal, ophthalmic and pulmonary manifestations. Symptoms can appear at any age and vary greatly between individuals even within the same family. Cardiovascular involvement is characterized by 1) progressive dilation of the aorta accompanied by an increased risk of aortic dissection, which affects prognosis; the aortic dilation can result in a leaky aortic valve; and 2) mitral insufficiency, which can be complicated by arythmias, endocarditis or cardiac insufficiency. Skeletal involvement is often the first sign of the disease and can include dolichostenomelia (excessive length of extremities), large size, arachnodactyly, joint hypermobility, scoliotic deformations, acetabulum protrusion, thoracic deformity (pectus carinatum or pectus excavatum), dolichocephaly of the anteroposterior axis, micrognathism or malar hypoplasia. Ophthalmic involvement results in axile myopia, which can lead to retinal detachment and lens displacement (ectopia or luxation are characteristic signs). Ocular complications, particularly lens ectopia, can lead to blindness. Cutaneous signs (vergetures), a risk of pneumothorax and dural ectasia can also occur. In the vast majority of cases, Marfan syndrome is caused by mutations of the FBN1 gene (15q21), which codes for fibrilline-1, a protein essential for connective tissues. Frontier forms have been identified that are secondary to mutations in the TGFBR2 gene located on chromosome 3, which codes for a TGF-beta receptor.

As used herein, the term “treatment” or “treat” refers to both prophylactic or preventive treatment as well as curative or disease modifying treatment, including treatment of patients at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular interval, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

In particular, the TREM-2 agonist of the present invention is particularly suitable for preventing ascending aorta rupture.

As used herein, the term “TREM-2” has its general meaning in the art and refers to the Triggering receptor expressed on myeloid cells 2. TREM-2 is variously referred to as TREM-2, TREM-2a, TREM-2b, TREM-2c, triggering receptor expressed on myeloid cells-2a, and triggering receptor expressed on monocytes-2. TREM-2 is a 230 amino acid membrane protein. TREM-2 is an immunoglobulin-like receptor primarily expressed on myeloid lineage cells, including without limitation, macrophages, dendritic cells, monocytes, Langerhans cells of skin, Kupffer cells, osteoclasts, and microglia. An exemplary amino acid sequence is represented by SEQ ID NO: 1. The extracellular domain of TREM-2 ranges from the amino acid residue at 19 to the amino acid residue at position 174 in SEQ ID NO:1.

>sp |Q9NZC2 | TREM-2 HUMAN Triggering receptor
expressed on myeloid cells 2 OS = Homo sapiens
OX = 9606 GN = TREM-2 PE = 1 SV = 1
SEQ ID NO: 1
MEPLRLLILLFVTELSGAHNTTVFQGVAGQSLQVSCPYDSMKHWGRRKAW
CRQLGEKGPCQRVVSTHNLWLLSFLRRWNGSTAITDDTLGGTLTITLRNL
QPHDAGLYQCQSLHGSEADTLRKVLVEVLADPLDHRDAGDLWFPGESESF
EDAHVEHSISRSLLEGEIPEPPTSILLLLACIFLIKILAASALWAAAWHG
QKPGTHPPSELDCGHDPGYQLQTLPGLRDT

As used herein, the term “TREM-2 agonist” refers to any compound, chemical, antibody, or peptide, naturally occurring or synthetic, that directly or indirectly increase one or more TREM-2 activities. In particular embodiment, the TREM-2 agonist directly bind to TREM-2 to increase one or more TREM-2 activities. The one or more TREM-2 activities are selected from the group consisting of: (a) TREM-2 binding to DAP12; (b) DAP12 phosphorylation; (c) activation of Syk kinase; (d) modulation of one or more pro-inflammatory mediators selected from the group consisting of IFN-β, IL-1α, IL-1β, TNF-α, IL-6, IL-8, CRP, CD86, MCP-1/CCL2, CCL3, CCL4, CCL5, CCR2, CXCL-10, Gata3, IL-20 family members, IL-33, LIF, IFN-gamma, OSM, CNTF, CSF-1, OPN, CD11c, GM-CSF, IL-11, IL-12, IL-17, IL-18, and IL-23, optionally wherein the modulation occurs in one or more cells selected from the group consisting of macrophages, M1 macrophages, activated M1 macrophages, M2 macrophages, dendritic cells, monocytes, osteoclasts, Langerhans cells of skin, Kupffer cells, and microglial cells; (e) recruitment of Syk to a DAP12/TREM-2 complex; (f) increasing activity of one or more TREM-2-dependent genes, optionally wherein the one or more TREM-2-dependent genes comprise nuclear factor of activated T-cells (NFAT) transcription factors; (g) increased survival of dendritic cells, macrophages, M1 macrophages, activated M1 macrophages, M2 macrophages, monocytes, osteoclasts, Langerhans cells of skin, Kupffer cells, microglia, M1 microglia, activated M1 microglia, and M2 microglia, or any combination thereof; and (h) modulated expression of one or more stimulatory molecules selected from the group consisting of CD83, CD86 MHC class II, CD40, and any combination thereof, optionally wherein the CD40 is expressed on dendritic cells, monocytes, macrophages, or any combination thereof, and optionally wherein the dendritic cells comprise bone marrow-derived dendritic cells. In some embodiments, the increase in one more TEM2 activities may be measured by any suitable in vitro cell-based assays or suitable in vivo model described herein or known in the art, for example, by utilizing a luciferase-based reporter assay to measure TREM-2-dependent gene expression, using Western blot analysis to measure increase in TREM-2-induced phosphorylation of downstream signaling partners, such as Syk, or by utilizing flow cytometry, such as fluorescence-activated cell sorting (FACS) to measure changes in cell surface levels of markers of TREM-2 activation. Any in vitro cell-based assays or suitable in vivo model described herein or known in the art may be used to measure interaction (e.g., binding) between TREM-2 and one or more TREM-2 ligands. The skilled in the art can easily determine whether a TREM-2 agonist enhances, increases or activates one or more TREM-2 activities.

In some embodiments, the TREM-2 agonist is an agonist TREM-2 antibody.

As used herein, the term “antibody” has its general meaning in the art and refers to an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, chimeric antibodies, humanized antibodies, human antibodies, fusion proteins comprising an antibody, and any other modified immunoglobulin molecule so long as the antibodies exhibit the desired biological activity. An antibody can be of any of the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy-chain constant domains referred to as alpha, delta, epsilon, gamma, and mu, respectively. The light chain includes two domains, a variable domain (VL) and a constant domain (CL). The heavy chain includes three (α, δ, γ) to five (μ, ε) domains, a variable domain (VH) and three to four constant domains (CH1, CH2, CH3 and CH4 collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from nonhypervariable or framework regions (FR) can participate to the antibody binding site or influence the overall domain structure and hence the combining site. CDRs refer to amino acid sequences which together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, typically includes six CDRs, comprising the CDR set from each of a heavy and a light chain V region. Framework Regions (FRs) refer to amino acid sequences interposed between CDRs. The residues in antibody variable domains are conventionally numbered according to a system devised by Kabat et al. This system is set forth in Kabat et al., 1987, in Sequences of Proteins of Immunological Interest, US Department of Health and Human Services, NIH, USA (hereafter “Kabat et al.”). This numbering system is used in the present specification. The Kabat residue designations do not always correspond directly with the linear numbering of the amino acid residues in SEQ ID sequences. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence. The CDRs of the heavy chain variable domain are located at residues 31-35B (H-CDR1), residues 50-65 (H-CDR2) and residues 95-102 (H-CDR3) according to the Kabat numbering system. The CDRs of the light chain variable domain are located at residues 24-34 (L-CDR1), residues 50-56 (L-CDR2) and residues 89-97 (L-CDR3) according to the Kabat numbering system.

As used herein, the term “agonist TREM-2 antibody” “activating TREM-2 antibody” is an antibody that induces (e.g., increases) one or more activities or functions of TREM-2 after the antibody binds to TREM-2. For example, the agonist TREME antibodies may have the correct epitope specificity that is compatible with receptor activation, as well as the ability to induce or retain receptor clustering on the cell surface. In addition, agonist anti-TREM-2 antibodies of the present disclosure may display the ability to bind TREM-2 without blocking simultaneous binding of one or more TREM-2 ligands. The anti-TREM-2 antibodies of the present disclosure may further display additive and/or synergistic functional interactions with one or more TREM-2 ligands. In some embodiments, enhancement of the one or more TREM-2 activities induced by binding of one or more TREM-2 ligands to the TREM-2 protein is measured on primary cells, including without limitation, dendritic cells, bone marrow-derived dendritic cells, monocytes, microglia, macrophages, neutrophils, NK cells, osteoclasts, Langerhans cells of skin, and Kupffer cells, or on cell lines, and the enhancement of the one or more TREM-2 activities induced by binding of one or more TREM-2 ligands to the TREM-2 protein is measured, for example, utilizing an in vitro cell assay. In some embodiments, anti-TREM-2 antibodies of the present disclosure have isotypes of human antibodies, such as IgG2, that have, due to their unique structure, an intrinsic ability to cluster receptors or retain receptors in a clustered configuration, thereby activating receptors such as TREM-2 without binding to an Fc receptor (e.g., White et al., (2015) Cancer Cell 27, 138-148).

In some embodiments, the agonist TREM-2 antibodies bind to human TREM-2 at an epitope within the extracellular domain of human TREM-2. In some embodiments, the agonist TREM-2 antibodies bind to human TREM-2 at an epitope within amino acids 19-174 of SEQ ID NO: 1. In some embodiments, the agonist TREM-2 antibodies bind to human TREM-2 at an epitope within amino acids 23-128 of SEQ ID NO: 1 or to an epitope within amino acids 131-148 of SEQ ID NO:1.

In some embodiments, the agonist TREM-2 antibodies of the invention do not specifically bind to human TREM1.

Agonist TREM-2 antibodies are well known in the art typically include those described in U.S. Pat. Nos. 10,508,148B2, 10,676,525B2, 11,084,875B2, 11,124,567B2, 11,186,636B2, WO2017062672, WO2018195506, WO2019055841, WO2019079529, WO2020055975, and WO2020121195. Other examples of agonist TREM-2 antibodies include those described in Fassler, M, Rappaport, M. S., Cuño, C. B. et al. Engagement of TREM-2 by a novel monoclonal antibody induces activation of microglia and improves cognitive function in Alzheimer's disease models. J Neuroinflammation 18, 19 (2021); Okuzono Y, Sakuma H, Miyakawa S, Ifuku M, Lee J, Das D, Banerjee A, Zhao Y, Yamamoto K, Ando T, Sato S. Reduced TREM-2 activation in microglia of patients with Alzheimer's disease. FEBS Open Bio. 2021 November; 11(11):3063-3080; and Ibach M, Mathews M, Linnartz-Gerlach B, Theil S, Kumar S, Feederle R, Brüstle O, Neumann H, Walter J. A reporter cell system for the triggering receptor expressed on myeloid cells 2 reveals differential effects of disease-associated variants on receptor signaling and activation by antibodies against the stalk region. Glia. 2021 May; 69(5):1126-1139.

In some embodiments, the agonist TREM-2 antibody of the present invention comprises a light chain variable region having complementarity determining regions CDRL1, CDRL2, and CDRL3, and a heavy chain variable region having complementarity determining regions CDRH1, CDRH2, and CDRH3, wherein CDRL1 comprises the amino acid sequence of: RASQSVSSNLA (SEQ ID NO:2); CDRL2 comprises the amino acid sequence of: GASTRAT (SEQ ID NO:3); CDRL3 comprises the amino acid sequence of: LQDNNFPPT (SEQ ID NO:4); CDRH1 comprises the amino acid sequence of: SWIG (SEQ ID NO:5); CDRH2 comprises the amino acid sequence of: IIYPGDADARYSPSFQG (SEQ ID NO: 6); and CDRH3 comprises the amino acid sequence of: RRQGIFGDALDF (SEQ ID NO:7).

In some embodiments, the agonist TREM-2 antibody of the present invention comprises a light chain having the amino acid sequence of SEQ ID NO:8 and a heavy chain having the amino acid sequence of SEQ ID NO:9.

> light chain
SEQ ID NO: 8
EIVMTQSPATLSVSPGERATLSCRASQSVSSNLAWFQQKPGQAPRLLIY
GASTRATGIPARFSGSGSGTEFTLTISSLQPEDFAVYYCLQDNNFPPTF
GQGTKVDIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSENRGEC
> heavy chain
SEQ ID NO: 9
EVQLVQSGAEVKKPGESLKISCKGSGYSFTSYWIGWVRQMPGKGLEWMG
ITYPGDADARYSPSFQGQVTISADKSISTAYLQWSSLKASDTAMYFCAR
RRQGIFGDALDFWGQGTLVTVSSASTKGPSVFPLAPSSKSTSGGTAALG
CLVKDYFPEPVTVSWNSGALTSGVHTFPAVLQSSGLYSLSSVVTVPSSS
LGTQTYICNVNHKPSNTKVDKKVEPKSCDKTHTCPPCPAPELLGGPSVE
LFPPKPKDTLMISRTPEVTCVVVDVSHEDPEVKFNWYVDGVEVHNAKTK
PCEEQYGSTYRCVSVLTVLHQDWLNGKEYKCKVSNKALPAPIEKTISKA
KGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYT
QKSLSLSPGK

In some embodiments, the agonist TREM-2 antibody of the present invention comprises a light chain variable region having complementarity determining regions CDRL1, CDRL2, and CDRL3, and a heavy chain variable region having complementarity determining regions CDRH1, CDRH2, and CDRH3, wherein CDRL1 comprises the amino acid sequence of: KSSQSLLYSSNQKNYLA (SEQ ID NO:10); CDRL2 comprises the amino acid sequence of: WASTRES (SEQ ID NO:11); CDRL3 comprises the amino acid sequence of: QQYYNYPFT (SEQ ID NO:12); CDRH1 comprises the amino acid sequence of: DYNIH (SEQ ID NO: 13); CDRH2 comprises the amino acid sequence of: YIYPKNGGTGYTQKFK (SEQ ID NO:14); and CDRH3 comprises the amino acid sequence of: RTARASWFAF (SEQ ID NO:15).

In some embodiments, the agonist TREM-2 antibody comprises a VH comprising the amino acid sequence of SEQ ID NO:16. In some embodiments, the agonist TREM-2 antibody comprises a VL comprising the amino acid sequence of SEQ ID NO:17. In some embodiments, the agonist TREM-2 antibody comprises a VH comprising the amino acid sequence of SEQ ID NO:16 and a VL comprising the amino acid sequence of SEQ ID NO: 17.

> VH Sequence
SEQ ID NO: 16
EVQLVQSGAEVKKPGESLKISCKGSGYTFTDYNIHWVRQMPGKGLEWMGY
IYPKNGGTGYTQKFKSQVTISVDNSISTAYLQWSSLKASDTAMYYCARRT
ARASWFAFWGQGTLVTVSS
> VL sequence
SEQ ID NO: 17
DIVMTQSPATLSVSPGERATLSCKSSQSLLYSSNQKNYLAWYQQKPGQAP
RVLIYWASTRESGIPARFSGSGSGTEFTLTISSLQSEDFAVYYCQQYYNY
PFTFGQGTKLEIK

In some embodiments, the TREM-2 agonist antibody of the present invention comprises a VH and a VL, wherein the VH comprises the same amino acid sequence as the VH of the antibody produced by the CGX-c hybridoma deposited at the ATCC® as deposit number PTA-125491 on Nov. 14, 2018.

As used herein, the term “therapeutically effective amount” refers to a sufficient amount of the TREM-2 agonist to treat Marfan Syndrome in the subject. It will be understood, however, that the total daily usage of the agent is decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidential with the specific agent; and like factors well known in the medical arts. For example, it is well within the skill of the art to start doses of the compound at levels lower than those required to achieve the desired therapeutic effect and to gradually increase the dosage until the desired effect is achieved. However, the daily dosage of the agent may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the agent for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

Typically, the TREM-2 agonist of the present invention is combined with pharmaceutically acceptable excipients, and optionally sustained-release matrices, such as biodegradable polymers, to form pharmaceutical compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. Typically, the pharmaceutical compositions contain vehicles, which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Sterile injectable solutions are prepared by incorporating the active ingredient at the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The invention will be further illustrated by the following figures and examples. However, these examples and figures should not be interpreted in any way as limiting the scope of the present invention.

### FIGURES

FIG. 1: A. qPCR quantification of Trem-2 mRNA in the ascending aorta of control (Fibrillin WT) or Marfan mice (Fibrillin mgR) at 10 weeks of age (Right, N=7-8/group). ***, P<0.001. B. qPCR quantification of Trem2 mRNA in the spleen of control (FibrillinWt/Wt) or MFS mice (Fibrillin mgR/mGr) at 8 weeks of age (Right, N=8-12/group

FIG. 2: qPCR quantification of Trem-2 mRNA in the ascending and the abdominal aorta of Marfan mice (Fibrillin mgR) at 10 weeks of age (Right, N=7-8/group). *, P<0.05.

FIG. 3: Representative pictures of ascending aorta sections of Fibrillin^{WT/WT}, Fibrillin^{mgR/mgR} mice Trem-2^{+/+} and Fibrillin^{mgR/mgR} mice Trem-2^{-/-} and quantification of ascending aorta diameter at 10 weeks of age. *, P<0.05; **, P<0.01.

FIG. 4: Survival curves of Fibrillin^{mgR/mgR}Trem-2^{+/+} (N=20) and Fibrillin mgR/mgR/Trem-2^{-/-} mice (N=10-14/group).

FIG. 5: A. Quantification of collagen content in the ascending aorta of Fibrillin^{mgR/mgR} Trem2^{+/+} and Fibrillin^{mgR/mgR} Trem2^{-/-} mice (N=7/group). Bar scale 50 um. B. Quantification of ascending aorta sections of Fibrillin^{mgR/mgR} Trem2^{+/+} and Fibrillin^{mgR/mgR} Trem2^{-/-} mice at 10 weeks of age, Cartography of MMP-2, -3, -9, and -13 activity in the aorta of Fibrillin^{mgR/mgR} Trem2^{+/+} and Fibrillin^{mgR/mgR} Trem2^{-/-} mice at 10 weeks of age. MMP activity was measured using MMPsense 680 (NEV 10126, PerkinElmer), images were acquired using a fluorescence molecular imaging system (FMT 2500™, VisEnMedical) (N=5). Bar scale 50 um **, P<0.01; ***, P<0.001.

FIG. 6: Five-week old Fibrillin^{mgR/mgR} mice were treated orally either by PBS or Ki20227, an inhibitor of CSF1-receptor with a pulse protocol (alternate one week of KI20227 treatment with 1 week of PBS) during 15 weeks. A. Quantification of CD68+ macrophage content in the ascending aorta. Bar scale 50 um. B. Monitoring of ascending aorta diameter using ultrasonography. C. survival monitoring.

FIG. 7: A. Quantification of CD68+ macrophages in the ascending aorta sections of Fibrillin^{mgR/mgR} Trem2^{+/+} and Fibrillin^{mgR/mgR} Trem2^{-/-} mice. (N=6/group, non-parametric test). Il6 and Il1b mRNA levels in the ascending aorta of 8-week old Fibrillin^{mgR/mgR} Trem2^{+/+} and Fibrillin^{mgR/mgR} Trem2^{-/-} mice (N=6/group). *, P<0.05.

### EXAMPLE

### TREM-2 is Expressed in the Aortic Wall of Marfan Mice.

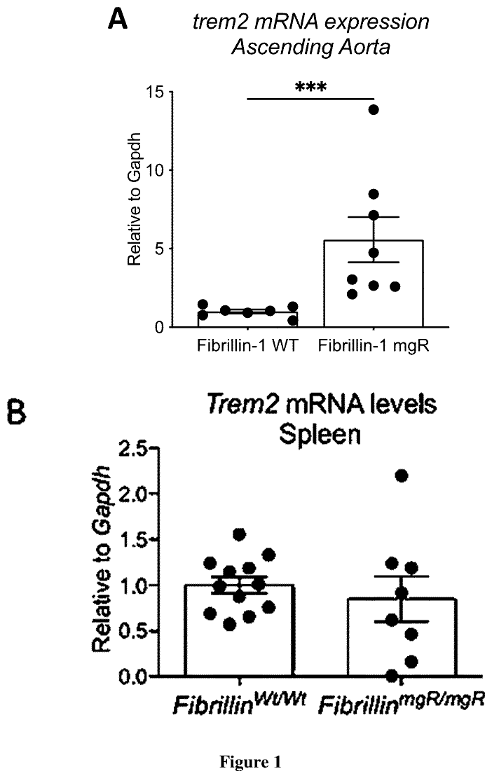

First, we analyzed by qPCR the gene expression of Trem2 in the whole aorta of fibrillin-1 hypomorphic, named Fibrillin mgR/mgR mice. This is a mouse model of haploinsufficiency that mimics Marfan syndrome and we compared it to littermate control mice (Fibrillin WT). We showed that Trem-2 m RNA levels are 5-fold higher in the aorta of fibrillin mGr/mGr mice compared to control mice at 10 weeks of age (FIG. 1A). Up-regulation of Trem2 transcripts was observed in the aorta, but not in other organs such as the spleen, supporting a specific vascular phenotype (FIG. 1B). Single-cell RNA sequencing (scRNA-seq) analysis showed higher total and Trem-2+ macrophages content in the aorta of Marfan mice, compared to control mice, TREM2 expression in the aortic wall being almost restricted to macrophage populations (data not shown). We also found that TREM-2 was specifically expressed by macrophages in the human aortic tissue from patients with MFS using scRNA-seq (data not shown).

### TREM-2 Expression in Higher in the Ascending Aorta

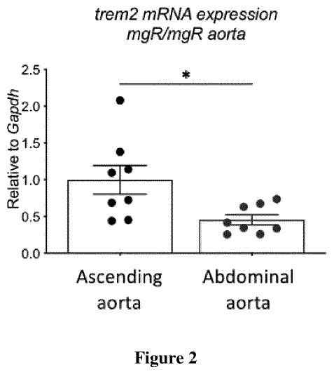

Next, in Fibrillin mgR/mgR mice, we compared by qPCR the levels of Trem-2 mRNA in 2 different aorta regions: the ascending aorta where aneurysm develops and the abdominal aorta. Interestingly, the levels of Trem2 transcripts were 2-fold higher in the ascending aorta (FIG. 2).

### 2. 3. TREM-2 Deficiency Aggravates Ascending Aorta Dilation in a Mouse Model of Marfan Syndrome.

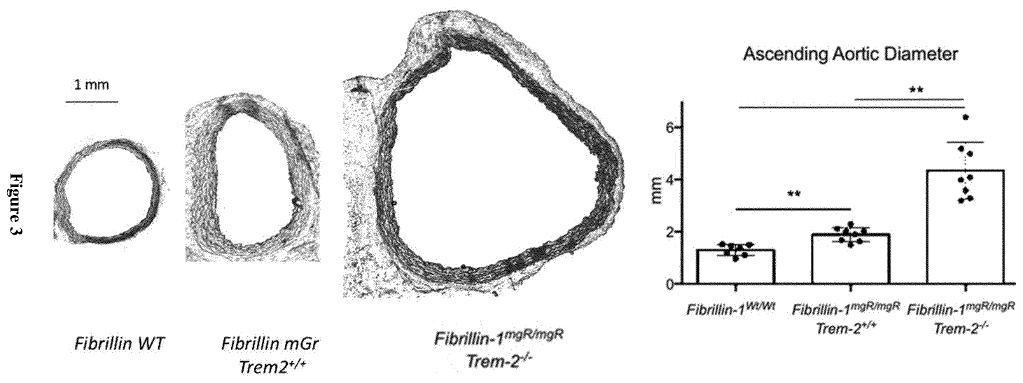

In order to study the role of TREM-2 in the aortic pathology of Marfan syndrome, we crossed Fibrillin mgR/mgR mice (Pereira et al. PNAS 1999) with Trem-2 deficient mice (Trem-2^{-/-}) (Seno, PNAS 2009), to generate Fibrillin^{mgR/mgR} mice Trem-2^{+/+} and Fibrillin^{mgR/mgR} mice Trem-2^{-/-}. We showed that Trem2 deficiency markedly aggravated MFS aortic disease with a 3-fold increase in ascending aorta dilation (n=7-8/group, FIG. 3)

### TREM-2 Deficiency Aggravates Lethal Aortic Rupture in a Mouse Model of Marfan Syndrome.

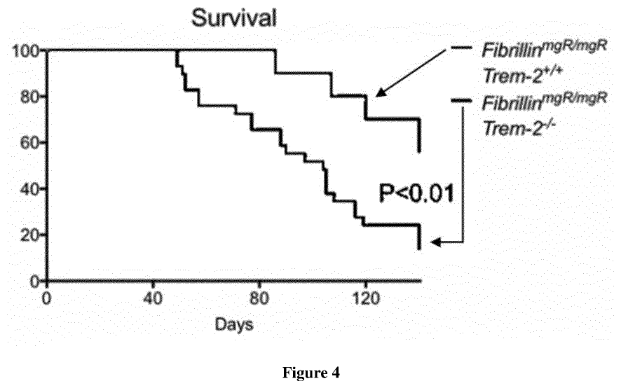

In order to study the role of TREM-2 in the complications of aortic pathology in Marfan syndrome, the animals (males and females) were followed and survival was recorded. As depicted in FIG. 4, we found that Trem-2 deficiency aggravates aortic aneurysm rupture and premature higher mortality in both males and females (n=10-14/group, FIG. 4), associated with aggravated non-cardiovascular abnormalities related to MFS, including skeleton disorders and rectal prolapse (observations and CT-scan).

### Extracellular Matrix Deleterious Remodeling

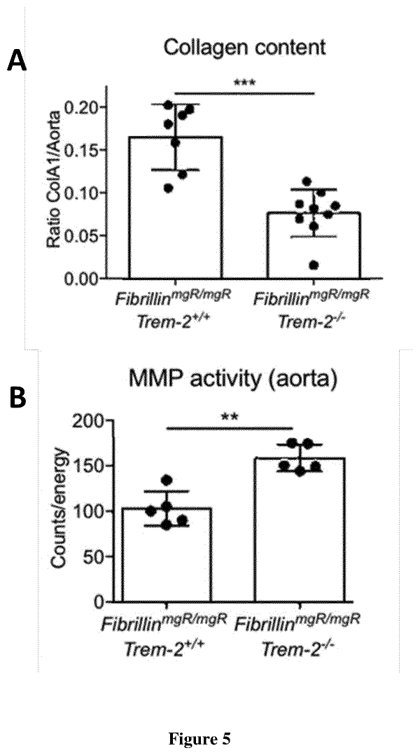

Several studies have reported alterations in the extracellular matrix in the MFS aortic wall, responsible for dilation and rupture. Here we showed that Trem2 deficiency aggravated deleterious aortic wall remodeling, with a decrease in local collagen content (FIG. 5A) associated with markedly increased local MMP-2, -3, -9, and -13 activity (FIG. 5B).

### Local Immune Responses

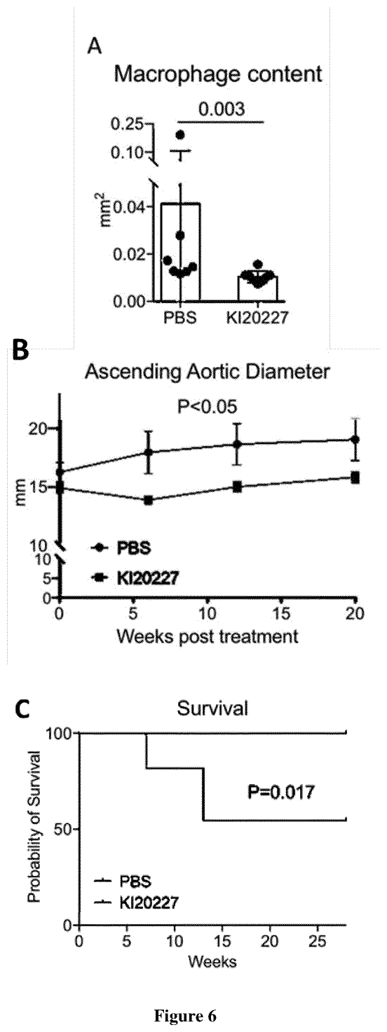

To evaluate the contribution of aortic resident macrophages to disease severity, Fibrillin^{mgR/mgR} mice have been treated by Ki20227, an inhibitor of macrophage colony stimulating factor 1 (CSF1) receptor tyrosine kinase. Pulse Ki20227 treatment depleted tissue macrophages (FIG. 6A) and limited both aorta dilation and rupture, supporting a pathogenic role of local macrophages on vascular deleterious remodelling (FIG. 6B-C) in MFS mice.

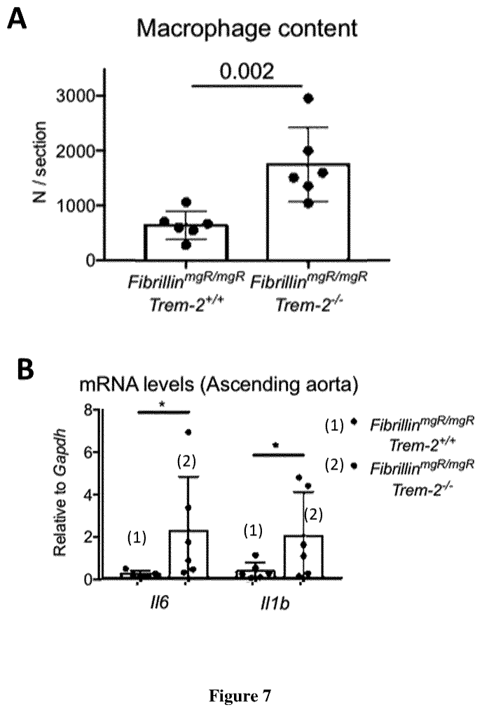

We found that Trem2 gene deletion was associated with a huge increase in macrophage content in both the media and adventitia (FIG. 7A), associated with a deviation of the local inflammatory response towards a pro-inflammatory phenotype (FIG. 7B).

### CONCLUSION

For the first time, we have demonstrated a critical role for TREM-2 in the pathophysiology of ascending aortopathy related to Marfan disease. Deletion of TREM-2 aggravates ascending aorta dilation and rupture. Stimulating TREM-2 receptor with peptide or agonistic monoclonal antibody represent a new therapeutic approach for Marfan syndrome.

### REFERENCES

Throughout this application, various references describe the state of the art to which this invention pertains. The disclosures of these references are hereby incorporated by reference into the present disclosure.

1. A method of treating Marfan Syndrome in a patient in need thereof comprising administering to the patient a therapeutically effective amount of a TREM-2 agonist.

2. The method of claim 1 wherein the TREM-2 agonist is suitable for preventing ascending aorta rupture.

3. The method of claim 1 wherein the TREM-2 agonist is an agonist TREM-2 antibody.

4. The method of claim 3 wherein the agonist TREM-2 antibody binds to human TREM-2 at an epitope within amino acids 19-174 of SEQ ID NO: 1.

5. The method of claim 3 wherein the agonist TREM-2 antibody binds to human TREM-2 at an epitope within amino acids 23-128 of SEQ ID NO: 1 or to an epitope within amino acids 131-148 of SEQ ID NO:1.

6. The method of claim 3 wherein the agonist TREM-2 antibody comprises a light chain variable region having complementarity determining regions CDRL1, CDRL2, and CDRL3, and a heavy chain variable region having complementarity determining regions CDRH1, CDRH2, and CDRH3, wherein CDRL1 comprises the amino acid sequence: RASQSVSSNLA (SEQ ID NO:2); CDRL2 comprises the amino acid sequence: GASTRAT (SEQ ID NO:3); CDRL3 comprises the amino acid sequence: LQDNNFPPT (SEQ ID NO:4); CDRH1 comprises the amino acid sequence: SWIG (SEQ ID NO:5); CDRH2 comprises the amino acid sequence: IIYPGDADARYSPSFQG (SEQ ID NO:6); and CDRH3 comprises the amino acid sequence: RRQGIFGDALDF (SEQ ID NO:7).

7. The method of claim 6 wherein the TREM-2 antibody comprises a light chain having the amino acid sequence of SEQ ID NO:8 and a heavy chain having the amino acid sequence of SEQ ID NO:9.

8. The method of claim 3 wherein the agonist TREM-2 antibody comprises a light chain variable region having complementarity determining regions CDRL1, CDRL2, and CDRL3, and a heavy chain variable region having complementarity determining regions CDRH1, CDRH2, and CDRH3, wherein CDRL1 comprises the amino acid sequence: KSSQSLLYSSNQKNYLA (SEQ ID NO:10); CDRL2 comprises the amino acid sequence: WASTRES (SEQ ID NO:11); CDRL3 comprises the amino acid sequence: QQYYNYPFT (SEQ ID NO:12); CDRH1 comprises the amino acid sequence: DYNIH (SEQ ID NO:13); CDRH2 comprises the amino acid sequence: YIYPKNGGTGYTQKFK (SEQ ID NO:14); and CDRH3 comprises the amino acid sequence: RTARASWFAF (SEQ ID NO:15).

9. The method of claim 8 wherein the agonist TREM-2 antibody comprises a VH comprising the amino acid sequence of SEQ ID NO:16 and/or a VL comprising the amino acid sequence of SEQ ID NO:17.

10. The method of claim 3 wherein the agonist TREM-2 antibody comprises a VH and a VL, wherein the VH comprises the same amino acid sequence as the VH of an antibody produced by the CGX-c hybridoma deposited at the ATCC® as deposit number PTA-125491.
